(19) United States
(12) Patent Application Publication
(10) Pub. No.: US 2011/0189312 A1
(43) Pub. Date: Aug. 4, 2011

(54) Compositions and Methods for Supporting Heat Shock Protein Function

(76) Inventors: Marvin A. Heuer, (US); Ken Clement, (US); Michele Molino, (US); Joseph Macdougall, (US); Phillip Apong, (US); Jason Peters, (US)

(21) Appl. No.: 13/019,811

(22) Filed: Feb. 2, 2011

Related U.S. Application Data
(62) Division of application No. 12/338,346, filed on Dec. 18, 2008, now abandoned.

(51) Int. Cl. A61K 36/79; A61K 36/65; A61K 31/7048; A61K 31/385; A61P 43/00

(52) U.S. Cl. 424/725; 514/27; 514/440

(57) ABSTRACT

Compositions and methods for promoting or maintaining protein accretion in cells, particularly in skeletal muscle cells, by supporting heat shock protein function. The compositions comprise glutamine and additional components directed at enhancing the activity of heat shock proteins.

### RELATED APPLICATIONS

The present application is related to, and claims benefit of priority to, the applicant's co-pending U.S. Patent Application Ser. No. 11/962,948, entitled “Compositions and methods for enhancing protein accretion in skeletal muscle” and Ser. No. 11/962,963, entitled “Compositions and methods for inducing the expression of heat shock proteins,” both filed Dec. 21, 2007, and the disclosures of both of which applications are hereby fully incorporated by reference.

### FIELD OF THE INVENTION

The present invention relates to a composition and method for supporting heat shock protein function in cells. Specifically, the present invention relates to a composition and method comprising a combination of at least one substance for activating or supporting heat shock protein function and glutamine, which act substantially simultaneously via differing mechanisms to increase heat shock protein function in cells, particularly heat shock protein 72 (HSP72) in skeletal muscle, to facilitate increased hypertrophy as a result of exercise.

### BACKGROUND OF THE INVENTION

When a mammalian cell is exposed to a sudden elevation in temperature the expression of most cellular proteins is decreased. However, some proteins, specifically heat shock proteins (HSP), show increased levels of expression when cells are subjected to elevated temperatures and other metabolic stresses. Examples of metabolic stresses which elicit elevated expression of heat shock proteins include: decreased glucose availability; increased intercellular calcium levels; oxidative stress, and decreased blood flow. HSPs are a highly conserved family of stress proteins present in all organisms from bacteria to humans. Heat shock proteins function as molecular chaperones to prevent protein aggregation and facilitate the folding of nascent proteins, particularly new peptides emerging from ribosomes, not only in conditions of stress but also under normal physiological conditions. Molecular chaperones recognize nascent proteins, predominantly via exposed hydrophobic residues, and bind selectively to those proteins to form relatively stable complexes. In these complexes, the protein is protected and able to fold into its functional form.

HSPs are categorized into families based on molecular weight. Among the many families of heat shock proteins, HSP72, the stress-inducible protein of the HSP70 family, is one of the best known endogenous factors protecting cells against tissue injury. Research of exercise-induced stress response has shown that exercise results in increased expression of HSP72 mRNA and subsequently in HSP72 protein.

Repetitive, forceful muscular contractions, i.e. physical exercise, cause changes in the expression patterns of genes and proteins. These changes can result in muscle adaptations such as muscle atrophy via muscle protein catabolism or muscle hypertrophy via muscle protein accretion. During hypertrophy, numerous nascent proteins are formed. An increase in the presence of molecular chaperones, such as HSP72, will act to enhance the stability of these nascent proteins until they can fold into their functional forms.

In situations of enhanced protein turnover, such as the environment in muscle following exercise as well as during recovery in the days following exercise, it would be advantageous for an individual to have a means of increasing the stability of rapidly forming proteins in order to reduce the catabolism of these non-folded proteins.

### SUMMARY OF THE INVENTION

The present invention relates to a composition and method for promoting or maintaining protein accretion in cells, particularly in skeletal muscle cells, by supporting heat shock protein function.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a composition and method of promoting or maintaining protein accretion in cells, particularly in skeletal muscle cells, by supporting heat shock protein function. Compositions and methods are presented that support heat shock protein function through multiple, non-mutually exclusive biological mechanisms.

As used herein, the term ‘subject’ refers to mammals and non-mammals. Mammals refers to any member of the Mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like.

The compositions according to the present invention may be included in foods, dietary supplements, nutraceuticals, medical foods, botanical drugs, homeopathic remedies, over-the-counter drugs, prescription drugs, and compounded drugs. Preferably, the compositions of the present invention are provided as nutritional compositions.

The acceptable routes of administration compatible with the various embodiments of the present invention include those well-known in the art and include: oral, rectal, and parenteral. As used here, the term ‘parenteral’ refers to methods of administration to that region outside of the digestive tract. Examples of parenteral routes of administration include, but are not limited to, subcutaneous, intramuscular or intravenous injection, and nasopharyngeal, mucosal or transdermal absorption. The preferred route is administration is oral and includes acceptable oral dosage forms commonly known in the art.

As used here, the term ‘acceptable oral dosage form’ would be known by one of skill in the art to include, for example, powder beverage mixes, liquid beverages, ready-to-eat bars, hard and soft capsules, tablets, caplets, and dietary gels.

Furthermore, the dosage forms of the present invention may be provided in accordance with customary processing techniques for any of the forms mentioned above. Additionally, the compositions set forth in the example embodiments herein may contain any appropriate number and type of excipients, as is well known in the art.

Material of the present disclosure that is of plant origin may be in the form of an extract. An extract, as used herein, is most simply a preparation derived from a plant source. Extracts suitable for use in the present invention may be produced by extraction methods as are known and accepted in the art such as alcoholic extraction, aqueous extractions, carbon dioxide extractions, for example. Extracts may be concentrated, removing most of the solvent and/or water. Such extracts are typically liquid but may subsequently be provided as a dry powder. Plant extracts may be standardized to a known compound present in the extract.

A plant extract may be made from the entire plant or any part thereof. Plant parts include leaves, stems, flowers, inflorescences, shoots, cotyledons, etc. The various parts may be dehydrated or used fresh. Often, the plant parts are washed before processing. Alternatively, forms of unprocessed, or raw, plants may be used in embodiments of the present invention. Such forms may be whole or part and may be fresh or dried. In preferred embodiments of the present invention, plant extracts are used.

As used herein, the phrase “promoting or maintaining protein accretion” refers to any act, process, or intervention which through any mechanism will act towards maintaining or increasing protein, particularly in skeletal muscle cells. Although the present invention is not to be limited by any theoretical explanation, it is herein understood that skeletal muscle protein breakdown (catabolism) and skeletal muscle protein buildup (anabolism) are biological processes that may occur simultaneously, each to varying degrees depending on multiple factors such as nutrient status and activity level. Although the present invention is not to be limited by any theoretical explanation, it is herein also understood that increase in skeletal muscle size (hypertrophy) is the result of, among other things, an increase in protein synthesis and that the propensity towards hypertrophy depends upon the net balance of catabolism versus anabolism.

As used herein, the term ‘heat shock proteins’, although the present invention is not to be limited by any theoretical explanation, is understood to encompass both proteins that are expressly labeled as such as well as other stress proteins, including homologs of such proteins that are expressed in the absence of stressful conditions. Thus, both inducible and constitutive heat shock proteins are included. Furthermore, as used herein, although the present invention is not to be limited by any theoretical explanation, the term ‘heat shock proteins’ is understood to encompass the mRNA species corresponding to expressly labeled heat shock proteins as well as other stress proteins, which are known to be translated into proteins. Still furthermore to be included within the term ‘heat shock proteins’ are factors that are known to regulate the expression or function of heat shock proteins such as heat shock transcription factor 1 (HSF1), a member of a family of transcription factors. When only either constitutive or inducible heat shock proteins are herein intended, they will be explicitly identified as such by reference as either constitutive or inducible.

As used herein, the phrase “supporting heat shock protein function” refers to any mechanism by which the biological role of heat shock proteins, as herein defined, is promoted, maintained, increased, enhanced, or in any way encouraged. The biological mechanisms to be promoted, maintained, increased, enhance, or in any way encouraged may include, but are not limited to: transcription of DNA encoding heat shock proteins, post-transcriptional modifications, translation of RNA encoding heat shock proteins into proteins, post-translational modifications serving to activate inactive heat shock proteins, and the transport of heat shock proteins, or components, thereof, to a location of activity. Also included are any like-biological mechanism affecting known regulators of heat shock proteins such as transcriptional regulators of heat shock proteins such as heat shock factor 1 (HSF1), for example. When only support of either constitutive or inducible heat shock protein function is herein intended, it will be explicitly identified as such by reference as either constitutive or inducible. Otherwise, the phrase “supporting heat shock protein function” herein refers to both constitutive and inducible

As used herein, the phrase “heat shock response facilitator” refers to any act, process, or intervention which through any mechanism will act towards maintaining or increasing inducible heat shock responses. As such, a “heat shock response facilitator” will support heat shock protein function as defined above for “supporting heat shock protein function”. HSP72 is the preferred mechanism through which heat shock response facilitators of the present invention act.

Glutamine

Glutamine is the most abundant amino acid found in the body and has important functions as a precursor for the synthesis of other amino acids.

Physical activity can deplete Glutamine levels, and as such, glutamine is often considered to be a ‘conditionally essential’ amino acid. A study examining the glutamine levels of groups involved in several different types of activities or sports found that powerlifters and swimmers had lower glutamine levels than cyclists and non-athletes, suggesting that high resistance load activities require increased amounts of glutamine.

Administration of glutamine has been shown to enhance protein expression and inhibit protein degradation in a condition-dependent manner. This regulation of protein turnover has been attributed to glutamine's affect on the expression of heat shock proteins in stressed conditions. Glutamine is capable of increasing the expression of heat shock proteins only in conditions of stress.

A study of the mechanism by which glutamine effects the expression of heat shock proteins has shown that glutamine does not affect the classical pathway of HSF1 activation. Instead, glutamine specifically modulates the transcriptional regulatory apparatus at the heat shock protein promoter, in a manner that is independent of HSF1.

Additionally, as used herein, ‘glutamine’ refers to glutamine derivatives such as esters, amides, and salts, as well as other derivatives, including derivatives having pharmacoproperties upon metabolism to an active form. Glutamine derivatives herein also include molecules, which at some time post-ingestion, yield glutamine. Glutamine derivatives are, for example, glutamic acid or glutamate, L-glutamine-ketoisocaproate, L-glutamine-alpha-ketoglutarate, and N-acetyl-L-glutamine.

Glutamine, as used herein, although the present invention is not to be limited by any theoretical explanation, is herein understood to include peptides, particularly di- and tri-peptides, containing at least one glutamine residue. A specific derivative of glutamine, a dipeptide of alanine and glutamine i.e. alanyl-glutamine, has been shown to induce heat shock protein and protect against vascular hyporeactivity. The preferred glutamine peptide is the dipeptide alanyl-glutamine.

Although the present invention is not to be limited by any theoretical explanation, it is herein understood by the inventors that inclusion of glutamine in a composition, will act to increase the production heat shock proteins, act as a coactivator, and modulate transcriptional regulatory machinery in the promoter region of the gene. Enhanced expression of heat shock proteins will act to increase protein accretion via increased stabilization of nascent proteins. The increased expression of chaperone proteins in working muscle stabilizes the large number of new proteins being synthesized by working muscle. This in turn leads to increased accumulation of contractile protein, i.e. muscle hypertrophy.

As used herein, a serving of the present composition comprises from about 1 mg to about 1.5 g of glutamine. More preferably, a serving of the present composition comprises from about 1 mg to about 1.0 g of glutamine. A serving of the present composition most preferably comprises from about 1 mg to about 750 mg of glutamine. The preferred derivative of glutamine is the dipeptide alanyl-glutamine.

Schisandrin Chinensis

Schisandrin B is a dibenzocyclooctadiene compound that is isolated from Schisandrae chinensis. Schisandrin B has been used to enhance the detoxification of xenobiotics in the liver and assist in liver regeneration. Recent studies have shown that schisandrin B can protect various organs from free-radical induced damage.

In a study using mice, administration of schisandrin B was shown to increase the production of HSP70. Treatment with schisandrin B produces oxidants via cytochrome p-450 metabolism, which act as mild stressors to induce HSP70 production.

Although the present invention is not to be limited by any theoretical explanation, it is herein understood by the inventors that inclusion of schisandrin Bin a composition, will act to increase the production of HSP72, by increasing the production of oxidants from cytochrome P-450 metabolism. Enhanced expression of HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, Le. muscle hypertrophy.

As used herein, a serving of the present composition comprises Schisandrae chinensis supplying schisandrin B. Preferably, the Schisandrae chinensis is in the form of an extract in the amount of from about 1 mg to about 500 mg. More preferably, a serving of the present composition comprises from about 10 mg to about 250 mg of Schisandrae chinensis. A serving of the present composition most preferably comprises from about 50 mg to about 150 mg of Schisandrae chinensis. Preferably, the amount of schisandrin B in a serving to the present invention is from about 0.001 mg to about 5 mg.

Paeoniflorin

Paeoniflorin is a major constituent of peony plants, such as Paeonia lactoflora, P. suffruticosa, P. obovata, and P. veitchii. The roots of peony plants have commonly been used in Chinese medicine to reduce fever and pain, stop bleeding, prevent infection, and as an antispasmodic.

In vitro studies showed that cells treated with paeoniflorin have enhanced levels of expression of heat shock proteins. Paeoniflorin treatment resulted in phosphorylation of HSF1 allowing HSF1 to translocate to the nucleus. Inside the nucleus phosphorylated HSF1 proteins combine to form granules (trimers) which have the ability to bind to the heat shock element region of inducible heat shock protein genes, thereby inducing transcription of these genes.

Additionally, as used herein, ‘paeoniflorin’ refers to paeoniflorin derivatives such as esters, amides, and salts, as well as other derivatives, including derivatives having pharmacoproperties upon metabolism to an active form. Paeoniflorin derivatives herein also include molecules, which at some time post-ingestion, yield paeoniflorin.

Although the present invention is not to be limited by any theoretical explanation, it is herein understood by the inventors that inclusion of paeoniflorin in a composition, will act to increase the expression of heat shock proteins, particularly HSP72, via directly activating HSF1. Paeoniflorin or derivatives of paeoniflorin will enhance the expression of heat shock proteins by increasing the phosphorylation and DNA-binding ability of HSF1 thereby facilitating the induction of heat shock proteins. Enhanced expression of heat shock proteins, particularly HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, i.e. muscle hypertrophy.

As used herein, a serving of the present composition comprises Paeonia species plant containing paeoniflorin. Preferably, the Paeonia species plant is in the form of an extract, the extract preferably comprising from about 1 mg to about 300 mg of paeoniflorin. More preferably, a serving of the present composition comprises from about 1 mg to about 150 mg of paeoniflorin. A serving of the present composition most preferably comprises from about 1 mg to about 75 mg of paeoniflorin.

Geranylgeranylacetone

Geranylgeranylacetone is an acyclic polyisoprenoid that has been used to protect gastric mucosa. Geranylgeranylacetone has been shown to activate transcription factors, particularly heat shock transcription factor HSF1, which are able to bind to DNA and induce transcription. HSF1 is normally suppressed since it is typically bound to the C-domain of constitutively active HSP70. Geranylgeranylacetone is able to bind to the C-domain of the HSP70 thereby causing HSF1 to dissociate. HSF1 is now able to undergo trimerization and be translocated to the nucleus, where it binds to the heat shock-responsive element in the promoter region of inducible HSP70 (i.e. HSP72) genes.

Recent experiments using cultured mouse skeletal cells, showed that treatment with geranylgeranylacetone up-regulated the expression of HSP72, and increased muscular protein content in a dose-dependent manner. Additionally geranylgeranylacetone was shown to facilitate the differentiation of myoblasts into myotubules.

Non-differentiated myoblasts, often referred to as satellite cells, are a small population of quiescent muscle precursor cells that occupy a “satellite” position immediately outside of muscle fibers. They are normally maintained in a quiescent state and become activated to fulfill roles of routine maintenance, repair and hypertrophy. Satellite cells are thought to be muscle-specific stem cells which are capable of producing large numbers of differentiated progeny as well as being capable of self-renewal. Such that satellite cells can fulfill their biological role, they must become activated, proliferate, differentiate and fuse to existing muscle cells. In this way, multinucleate muscle fibers are maintained or increased in size in response to stimuli.

Additionally, as used herein, ‘geranylgeranylacetone’ refers to geranylgeranylacetone derivatives such as esters, amides, and salts, as well as other derivatives, including derivatives having pharmacoproperties upon metabolism to an active form. Geranylgeranylacetone derivatives herein also include molecules, which at some time post-ingestion, yield geranylgeranylacetone.

Although the present invention is not to be limited by any theoretical explanation, it is herein understood by the inventors that inclusion of geranylgeranylacetone in a composition, will act to increase the expression of heat shock proteins, particularly HSP72, via directly activating HSF1. Enhanced expression of heat shock proteins, particularly HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, i.e. muscle hypertrophy.

Additionally, although the present invention is not to be limited by any theoretical explanation, it is herein understood by the inventors that administration of geranylgeranylacetone will have the added benefit of facilitating the differentiation of myoblasts to myofibers. These myofibers fuse with existing muscle cells thereby increasing the size of the muscle cells and ultimately muscle tissue.

As used herein, a serving of the present composition comprises from about 1 mg to about 300 mg of geranylgeranylacetone. More preferably, a serving of the present composition comprises from about 25 mg to about 150 mg of geranylgeranylacetone. A serving of the present composition most preferably comprises from about 25 mg to about 75 mg of geranylgeranylacetone.

Alpha Lipoic Acid

Alpha lipoic acid is a co-enzyme found in the cellular energy-producing structures, the mitochondria. Moreover, alpha lipoic acid works in synergy with vitamins C and E as an antioxidant in both water- and fat-soluble environments. As used herein, derivatives of alpha lipoic acid also includes derivatives of alpha lipoic acid such as esters, and amides, as well as other derivatives, including derivatives that become active upon metabolism.

Primarily as an antioxidant, alpha lipoic acid has been demonstrated to have efficacy as a protective against diabetic neuropathy; a benefit mediated by stimulating a heat shock response including HSF1 and HSP72.

Additionally, as used herein, ‘alpha lipoic acid’ refers to alpha lipoic acid derivatives such as esters, amides, and salts, as well as other derivatives, including derivatives having pharmacoproperties upon metabolism to an active form. Alpha lipoic acid derivatives herein also include molecules, which at some time post-ingestion, yield alpha lipoic acid. Alpha lipoic acid derivatives are, for example, calcium alpha lipoic acid, sodium alpha lipoic acid, and alpha lipoic acid tromethamine.

Although the present invention is not to be limited by any theoretical explanation, it is herein understood by the inventors that inclusion of alpha lipoic acid or derivatives of alpha lipoic acid in a composition, will act to increase the expression of heat shock proteins, particularly HSF1 and HSP72. Enhanced expression of heat shock proteins, particularly HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, i.e. muscle hypertrophy.

As used herein, a serving of the present composition comprises from about 1 mg to about 250 mg of alpha lipoic acid or derivatives of alpha lipoic acid. More preferably, a serving of the present composition comprises from about 1 mg to about 100 mg of alpha lipoic acid or derivatives of alpha lipoic acid. A serving of the present composition most preferably comprises from about 10 mg to about 50 mg of alpha lipoic acid or derivatives of alpha lipoic acid.

Creatine

Creatine is a naturally occurring amino acid derived from the amino acids glycine, arginine, and methionine. Although it is found in meat and fish, it is also synthesized by humans. Creatine is predominantly used as a fuel source in muscle. About 65% of creatine is stored in muscle as phosphocreatine (creatine bound to a Phosphate molecule). Muscular contractions are fueled by the dephosphorylation of adenosine triphosphate (ATP) to produce adenosine diphosphate (ADP) and without a mechanism to replenish ATP stores, the supply of ATP would be rapidly consumed. Phosphocreatine, which is generated from the phosphorylation of creatine by the enzyme creatine kinase, serves as a major source of phosphate from which ADP is regenerated to ATP.

Research indicates that the constitutive HSP70 family member, HSC70 is present in an inactive polymerized form that upon stimulation de-polymerized into predominantly more active monomeric form. Of particular significance is that the presence of Creatine Kinase or phosphocreatine contributes to the conversion of polymerized HSC70 to monomeric HSC70.

As used herein, ‘creatine’ refers to derivatives of creatine such as esters, amides, and salts, as well as other derivatives, including derivatives having pharmacoproperties upon metabolism to an active form. Creatine derivatives herein also include molecules, which at some time post-ingestion, yield creatine. Creatine derivatives are, for example, creatine ethyl ester, creatine alpha ketoglutarate creatine pyroglutamate, creatine pyruvate, and creatine taurinate.

Although the present invention is not to be limited by any theoretical explanation, it is herein understood by the inventors that inclusion of creatine or derivative of creatine in a composition, will act to increase portion of active HSC70, modulating the de-polymerization of HSC70 by acting as a substrate for Creatine Kinase to support the production of phosphocreatine. Increased monomeric HSC70 will act to increase protein accretion via increased stabilization of nascent proteins. The increased presence of chaperone proteins in working muscle is important in order to stabilize the large number of new proteins being synthesized by working muscle, leading to increased accumulation of contractile protein, i.e. muscle hypertrophy.

As used herein, a serving of the present composition comprises from about 0.5 g to about 5 g of creatine or derivative of creatine. More preferably, a serving of the present composition comprises from about 1 g to about 3.5 g of creatine or derivative of creatine. A serving of the present composition most preferably comprises from about 1.5 g to about 3 g of creatine or derivative of creatine.

The composition of the present invention may be administered in a dosage form having controlled release characteristics, e.g. time-release. Furthermore, the controlled release may be in forms such as a delayed release of active constituents, gradual release of active constituents, or prolonged release of active constituents. Such active constituents release strategies extend the period of bioavailability or target a specific time window for optimal bioavailability. Advantageously the composition may be administered in the form of a multi-compartment capsule which combines both immediate release and time-release characteristics. Individual components of the composition may be contained in differential compartments of such a capsule such that the specific components may be released rapidly while others are time-dependently released. Alternatively, a uniform mixture of the various components of the present invention may be divided into both immediate release and time-release compartments to provide a multi-phasic release profile.

According to various embodiments of the present invention, the composition may be consumed in any form. For instance, the dosage form of the composition may be provided as, e.g., a powder beverage mix, a liquid beverage, a ready-to-eat bar or drink product, a capsule, a liquid capsule, a soft-gel capsule, a tablet, a caplet, or as a dietary gel. The preferred dosage form of the present invention is as a caplet.

Furthermore, the dosage form of the composition may be provided in accordance with customary processing techniques for herbal and compositions in any of the forms mentioned above. Additionally, the compositions set forth in the example embodiment herein may contain any appropriate number and type of excipients, as is well known in the art.

The present compositions or those similarly envisioned by one of skill in the art, may be utilized in methods to promote or maintain protein accretion in cells, particularly in skeletal muscle cells, by supporting heat shock protein function, thereby increasing hypertrophy as a result of exercise.

In one embodiment, the present invention provides for compositions and methods for promoting or maintaining protein accretion in cells, particularly in skeletal muscle cells, by supporting heat shock protein function, particularly inducible heat shock proteins. Components of compositions and methods provided in accordance with this embodiment are directed towards inducible heat shock proteins which are modulated, in part, by HSF1. Not wishing to be bound by theory, it is believed that glutamine or derivative of glutamine, which act via mechanism independent of HSF1, together with at least one additional component known to support inducible heat shock protein function via HSF1, will act to promote or maintain protein accretion in cells, particularly in skeletal muscle cells via synergistic and distinct mechanisms.

In another embodiment, the present invention provides for compositions and methods for promoting or maintaining protein accretion in cells, particularly in skeletal muscle cells, by supporting both inducible and constitutive heat shock protein function. In addition to glutamine supporting the activity of inducible heat shock proteins independent of HSF1, and another component supporting the activity of inducible heat shock proteins via HSF1, creatine or a derivative of creatine is included to support the activity of constitutive heat shock proteins.

In addition to the foregoing, compositions of the present invention include formulations further comprising additional active ingredients and/or inactive ingredients, including solvents, diluents, suspension aids, thickening or emulsifying agents, sweeteners, flavorings, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and which may also be suitable for use in formulations of the present invention. Except insofar as any conventional carrier medium is incompatible with the ingredients of the invention, such as by producing any undesirable effect or otherwise interacting in a deleterious manner with any other ingredient(s) of the formulation, its use is contemplated to be within the scope of this invention.

Although the following examples illustrate the practice of the present invention in various embodiments, the examples should not be construed as limiting the scope of the invention. Other embodiments will be apparent to one of skill in the art from consideration of the specifications and examples.

### EXAMPLES

### Example 1

A composition comprising the following ingredients per serving are prepared for consumption as four caplets, to be taken twice daily:
about 500 mg of L-glutamine and about 100 mg of Schisandra chinensis fruit extract.

### Example 2

A composition comprising the following ingredients per serving are prepared for consumption as five caplets, to be taken twice daily:
about 5 mg of L-glutamine, about 100 mg of Schisandra chinensis fruit extract, and about 100 mg of Paeonia lactiflora root extract (standardized to 10% paeoniflorin).

### Example 3

A composition comprising the following ingredients per serving are prepared for consumption as six caplets, to be taken twice daily with one serving being taken prior to exercise:
about 10 mg of L-glutamine, about 100 mg of Schisandra chinensis fruit extract, and about 3 g of creatine monohydrate.

### Example 4

A composition comprising the following ingredients per serving are prepared for consumption as three caplets, to be taken twice daily with one serving being taken prior to exercise:
about 500 mg of L-glutamine and about 40 mg of geranylgeranylacetone.

Extensions and Alternatives

In the foregoing specification, the invention has been described with respect to specific embodiments thereof; however, it will be evident that various modifications and changes may be made thereto without departing from the broader spirit and scope of the invention.

All publications which are cited herein are hereby specifically incorporated by reference into the disclosure for the teachings for which they are cited.

What is claimed:

1. A composition for supporting heat shock protein function in cells, comprising;
glutamine; and
one other heat shock response facilitator.

2. The composition of claim 1, wherein the one other heat shock response facilitator is selected from the group consisting of: Schisandrae chinensis, Paeonia species plant, geranylgeranylacetone, and alpha lipoic acid.

3. The composition of claim 1, wherein the one other heat shock response facilitator is Schisandrae chinensis.

4. A composition for supporting heat shock protein function in cells, comprising;
glutamine;
creatine; and
one other heat shock response facilitator.

5. The composition of claim 4, wherein the one other heat shock response facilitator is selected from the group consisting of: Schisandrae chinensis, Paeonia species plant, geranylgeranylacetone, and alpha lipoic acid.

6. The composition of claim 4, wherein the one other heat shock response facilitatoris Schisandrae chinensis.

7. A method of supporting heat shock protein function in cells comprising the step of administering to a subject a composition comprising;
glutamine; and
one other heat shock response facilitator.

8. The method of claim 7, wherein the one other heat shock response facilitator is selected from the group consisting of: Schisandrae chinensis, Paeonia species plant, geranylgeranylacetone, and alpha lipoic acid.

9. The method of claim 7, wherein the one other heat shock response facilitatoris Schisandrae chinensis.

10. A method of supporting heat shock protein function in cells comprising the step of administering to a subject a composition comprising;
glutamine;
creatine; and
one other heat shock response facilitator.

11. The method of claim 10, wherein the one other heat shock response facilitator is selected from the group consisting of: Schisandrae chinensis, Paeonia species plant, geranylgeranylacetone, and alpha lipoic acid.

12. The method of claim 10, wherein the one other heat shock response facilitator is Schisandrae chinensis.

13. A composition comprising:
glutamine; and
Schisandrae chinensis.

14. The composition of claim 13, further comprising creatine.

15. The composition of claim 14, wherein:
the amount of glutamine is from about 1 mg to about 1.5 g;
the amount of Schisandrae chinensis is from about 1 mg to about 500 mg; and
the amount of creatine or a derivative of creatine is from about 0.5 g to about 5 g.

16. The composition of any of claims 13 to 15, wherein said composition is a nutritional composition.
